(19) United States
(12) Patent Application Publication
(10) Pub. No.: US 2017/0101366 A1
(43) Pub. Date: Apr. 13, 2017

(54) PROCESS FOR MAKING DI-FUNCTIONAL MOLECULES WITH CONCURRENT LIGHT PARAFFIN UPGRADING

(71) Applicant: ExxonMobil Research and Engineering Company, Annandale, NJ (US)

(72) Inventor: Kun Wang, Bridgewater, NJ (US)

(21) Appl. No.: 15/387,977

(22) Filed: Dec. 22, 2016

Related U.S. Application Data
(63) Continuation-in-part of application No. 14/956,477, filed on Dec. 2, 2015.
(60) Provisional application No. 62/092,485, filed on Dec. 16, 2014.

(51) Int. Cl. C07C 253/30; C07C 407/00; C07C 17/281; C07C 51/353; C07C 209/68; C07C 201/12; C07C 29/32; C07C 29/48

(52) U.S. Cl. CPC C07C 253/30; C07C 29/32; C07C 407/00; C07C 29/48; C07C 51/353; C07C 209/68; C07C 201/12; C07C 17/281

(57) ABSTRACT

An integrated process for making di-functional or multi-functional molecules with concurrent light paraffin upgrading is disclosed. The process involves three primary steps: (1) oxidation of an iso-paraffin to alkyl hydroperoxide and alcohol; (2) converting the alkyl hydroperoxide and alcohol to dialkyl peroxide; and (3) coupling functional molecules into di-functional or multi-functional molecules using the dialkyl peroxide as a radical initiator, while the dialkyl peroxide is converted to a tertiary alcohol. The functional molecules include any functional molecule R—X, where R is a hydrocarbyl group and X is a functional group such as —OH, —CN, —C(O)OH, —NH—, or the like.

### CROSS-REFERENCE TO RELATED APPLICATIONS

This application is a continuation-in-part of copending U.S. Ser. No. 14/956,477, tiled Dec. 2, 2015, now allowed, which claims the benefit of provisional U.S. Ser. No. 62/092,485, filed on Dec. 16, 2014, each of which is incorporated by reference herein in its entirety.

### BACKGROUND

The present disclosure relates to an integrated process for making di-functional or multi-functional molecules with concurrent light paraffin upgrading.

Di-functional products such as diols, di-acids, di-nitrites, and di-amines are valuable chemicals, and are building blocks for a variety of high performance materials. Di-functional molecules are difficult to make, however, often involving complicated multi-step processes with limited per pass yield. For example, diols can be manufactured from olefins via dihydroxylation using one of several well-known complex mechanisms. For instance, ethylene glycol [HOCH₂—CH₂OH] formation involves ethylene epoxidation to ethylene oxide, followed by hydrolysis of ethylene oxide. The selectivity for the ethylene epoxidation step is generally 70-75 mol %, with the rest of the ethylene over-oxidized to CO₂. Other examples of di-functional products include succinic acid [HO(O)CCH₂—CH₂C(O)OH], succinonitrile [NCCH₂—CH₂CN], and ethylene diamine [H₂NCH₂—CH₂NH₂].

There remains a need for an alternative route to creating di-functional materials using readily available starting materials. Light paraffins (C2-C5), in particular, are increasingly available in the North America region. It is thus desirable to have a process for upgrading these abundant light paraffins to higher value molecules, while at the same time enabling formation of di-functional molecules.

### SUMMARY

We have now found a novel integrated process for making di-functional or multi-functional molecules with concurrent light paraffin upgrading. In a first embodiment of the present disclosure, the process involves: (1) oxidation of an iso-paraffin to alkyl hydroperoxide and alcohol; (2) converting the alkyl hydroperoxide and alcohol to dialkyl peroxide; (3) coupling functional molecules into di-functional or multi-functional molecules using the dialkyl peroxide as a radical initiator, while the dialkyl peroxide is converted to a tertiary alcohol. The net reaction is thus conversion of functional molecules to di-functional molecules using iso-paraffin and air, with the iso-paraffin being upgraded to a tertiary alcohol.

In another embodiment of the present disclosure, the process involves (1) oxidation of iso-butane to t-butyl hydroperoxide and t-butyl alcohol; (2) converting the t-butyl hydroperoxide and the t-butyl alcohol to di-t-butyl peroxide; (3) coupling functional molecules into di-functional or multi-functional molecules using the di-t-butyl peroxide as a radical initiator, while the di-t-butyl peroxide is converted to t-butyl alcohol. The net reaction is thus conversion of functional molecules to di-functional or multi-functional molecules using iso-butane and air, with the iso-butane being upgraded to t-butyl alcohol.

### BRIEF DESCRIPTION OF FIGURES

FIG. 1 is a GC-MS trace for the reaction products of the Example (EG=ethylene glycol).

FIG. 2 is the GC-MS trace of FIG. 1, but zoomed in to the region containing ethylene glycol.

### DETAILED DESCRIPTION

All numerical values within the detailed description and the claims herein are modified by “about” or “approximately” the indicated value, and take into account experimental error and variations that would be expected by a person having ordinary skill in the art. The present disclosure relates to an integrated process for making di-functional or multi-functional molecules with concurrent light paraffin upgrading. The process of the present disclosure involves three primary steps: (1) oxidation of an iso-paraffin to alkyl hydroperoxide and alcohol; (2) converting the alkyl hydroperoxide and alcohol to dialkyl peroxide; and (3) coupling functional molecules into di-functional or multi-functional molecules using the dialkyl peroxide as a radical initiator, while the dialkyl peroxide is converted to a tertiary alcohol. The net reaction is thus conversion of functional molecules to di-functional or multi-functional molecules using iso-paraffin and air, with the iso-paraffin being upgraded to a tertiary alcohol. The functional molecules include any functional molecule R—X, where R is a hydrocarbyl group and X is a functional group such as halogens, —OH, —CN, —C(O)OH, —NH—, or the like.

In a preferred embodiment of the present disclosure, the iso-paraffin feedstock is iso-butane. The process proceeds as described generally above: (1) oxidation of iso-butane to t-butyl hydroperoxide and t-butyl alcohol; (2) converting the t-butyl hydroperoxide and the t-butyl alcohol to di-t-butyl peroxide; (3) coupling functional molecules into di-functional or multi-functional molecules using the di-t-butyl peroxide as a radical initiator, while the di-t-butyl peroxide is converted to t-butyl alcohol. The net reaction is thus conversion of functional molecules to di-functional or multi-functional molecules using iso-butane and air, with the iso-butane being upgraded to t-butyl alcohol.

The chemistry of Steps 1-3 with respect to iso-butane feed is shown below in corresponding Equations 1-3:

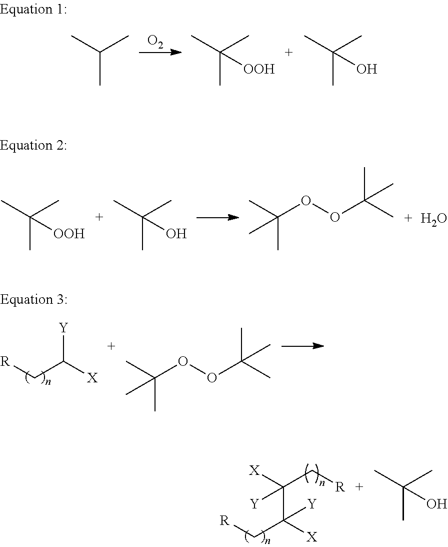

Steps 1 and 2 have been previously described with respect to mixed paraffinic feedstocks in applicant's co-pending application, U.S. Publ. App. No. 2016/0168048, incorporated by reference herein in its entirety. U.S. Publ. App. No. 2016/0168048 describes a process to convert light paraffins to heavier hydrocarbons generally, for example, distillates and lubricant base stocks, using coupling chemistry analogous to Steps 1 and 2 described above. Whereas U.S. Publ. App. No. 2016/0168048 is directed to mixed paraffinic feed to create distillates and lubricant base stocks, the present disclosure utilizes analogous coupling chemistry to create di-functional or multi-functional products utilizing functional molecules as feedstock.

Iso-butane oxidation in Step 1/Equation 1 is well-established commercially for making t-butyl hydroperoxide (TBHP) for propylene oxide manufacture, with variants of the process also described, for example, in U.S. Pat. No. 2,845,461; U.S. Pat. No. 3,478,108; U.S. Pat. No. 4,408,081 and U.S. Pat. No. 5,149,885. EP 0567336 and U.S. Pat. No. 5,162,593 disclose co-production of TBHP and t-butyl alcohol (TBA). As TBA is another reactant used in Step 2 of the present disclosure, the present inventive process scheme utilizes Step 1 as a practical source of these two reactants. Air (˜21% oxygen), a mixture of nitrogen and oxygen containing 2-20 vol % oxygen, or pure oxygen, can be used for the oxidation, as long as the oxygen-to-hydrocarbon vapor ratio is kept outside the explosive regime. Preferably air is used as the source of oxygen. Typical oxidation conditions for Step 1 of the present disclosure are: 110-150° C. (preferably 130 to 140° C., at a pressure of about 300-800 psig (preferably about 450-550 psig), with a residence time of 2-24 hours (preferably 6-8 h), to give a targeted conversion of 15%-70% (preferably 30-50%). Selectivity to TBHP of 50-80% and to TBA of 20-50% is typical.

In Step 2/Equation 2, the conversion of the TBHP and TBA to di-t-butyl peroxide (DTBP) is performed using an acid catalyst. For example, U.S. Pat. No. 5,288,919 describes the use of an inorganic heteropoly and/or isopoly acid catalyst (such as for the reaction of TBA with TBHP. The conjoint production of DTBP and TBA from TBHP is also described in U.S. Pat. No. 5,345,009. A preferred configuration for the present disclosure uses reactive distillation where product water is continuously removed as overhead by-product. Typical reaction temperature is in the range of 50-200° C., preferably 60-150° C., more preferably 80-120° C. The TBHP to TBA mole ratio is in the range of 0.5-2, preferably 0.8-1.5, more preferably 0.9-1.1. The reaction can be performed with or without a solvent. Suitable solvents comprise hydrocarbons having a carbon number greater than 3, such as paraffins, naphthenes, or aromatics. Conveniently, the unreacted iso-butane from Step 1 can be used as solvent for Step 2. Pressure for the reaction is held at appropriate ranges to ensure the reaction occurs substantially in the liquid phase, for example, 0-300 psig, preferably 5-100 psig, more preferably 15-50 psig. An acid catalyst such as Amberlyst™ resin, Nafion™ resin, aluminosilicates, acidic clay, zeolites (natural or synthetic), silicoaluminophosphates (SAPO), heteropolyacids, acidic oxides such as tungsten oxide on zirconia, molybdenum oxide on zirconia, sulfunated zirconia, liquid acids such sulfuric acid, or acidic ionic liquids may be used in Step 2/Equation 2 to promote the conversion of TBHP and TBA into DTBP.

In Step 3/Equation 3, DTBP is introduced to a coupling reactor to initiate free radical coupling of a feedstock comprising functional molecules, utilizing DTBP as a radical coupling reagent. The feedstock in Equation 3 includes a wide range of functional molecules, R—(CH₂)ₙ—CHXY, where —X or —Y or both can be a functional group. When only one of —X or —Y is a functional group, the other is hydrogen. Examples of functional groups include halogens (—F, —Cl, —Br, or —I), —OH (hydroxyl), —CN (cyano), —C(O)OH (carboxylic), —NHR′ (amino, where R′ can be hydrogen or a hydrocarbyl group), —SH (mercapto), —NO2 (nitro), —OSO₃H (sulfonato), —OPO3H (phosphato), —OBH (borato), and the like. R may be selected from hydrogen, hydrocarbyl (either linear or cyclic with a carbon number in the range of 1-40, preferably 1-10, and more preferably 1-4), or a functional group similar to those defined for —X and —Y. n is an integer in the range of 0-30. in the case when n=0 and R=H, examples of products and their corresponding feeds include ethylene glycol (from methanol feed), succinic acid (from acetic acid feed), succinonitrile (from acetonitrile feed), ethylene diamine (from methyl amine feed), 1,2-dinitroethane (from nitromethane feed), and 1,2-dichloroethane (from methyl chloride feed). In cases where n>0, isomers of di-functional or multi-functional products can be formed by connecting the different carbon atoms in the starting functional molecules. Different functional molecules can be used as feed to prepare a large variety of di-functional or multi-functional products. For example, the same type of functional materials with different n, or different types of functional molecules with different functional groups (same or different n) can be used as feed for Equation 3.

Typical reaction conditions for Step 3 of the present disclosure are: 100-170° C. (preferably about 145-155° C.), with pressure maintained to ensure that the feeds stay in the liquid or supercritical phase, typically 100-1500 psig (preferably about 500-1200 psig). Residence time is normally in the range of 2-24 hours (preferably 4-16 hours). Complete conversion of DTBP is normally achieved in this step. The molar ratio of DTBP to feedstock to be coupled is in the range of about 0.01-100, preferably in the range of about 0.05-10, and more preferably in the range of 0.1-2. Following, Step 3, the mixed product stream is fractionated, with unreacted feedstock being recycled to the coupling reactor, TBA and byproduct acetone being removed, and di-functional or multi-functional products recovered.

The overall reaction stoichiometry from Equations 1-3 is shown below in Equation 4:

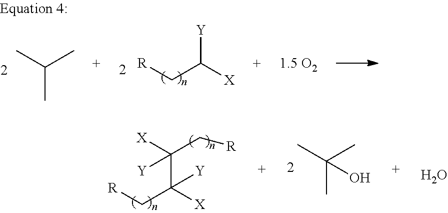

The net effect of Equations 1-3 is the coupling of functional molecules to di-functional or multi-functional products, using iso-butane as an oxygen carrier, while iso-butane is converted to t-butyl alcohol, which is itself an upgraded product from iso-butane. Depending on the nature of the iso-paraffin feed, the resulting alcohol can be used as a high octane blend for gasoline (e.g. t-butyl alcohol from iso-butane and 2-methyl-2-butanol from iso-pentane). Alternatively, the alcohols can be converted to olefins via dehydration (e.g., iso-butylene), or etherified with an alcohol such as methanol or ethanol to use as a gasoline blend (e.g., MTBE or ETBE from iso-butane)

### EXAMPLE

In order to provide a better understanding of the foregoing disclosure, the following non-limiting example is offered. Although the example may be directed to specific embodiments, they are not to be viewed as limiting the disclosure in any specific respect.

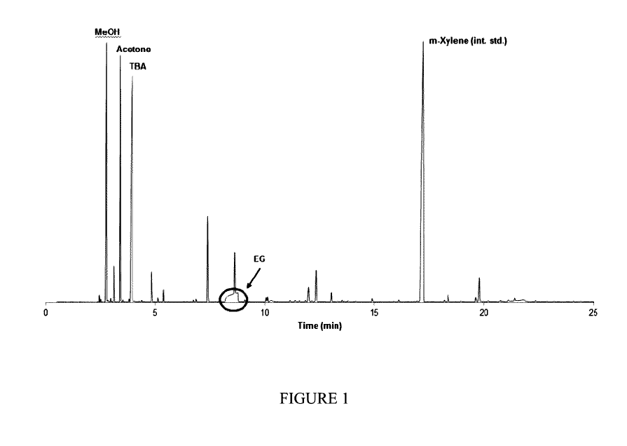

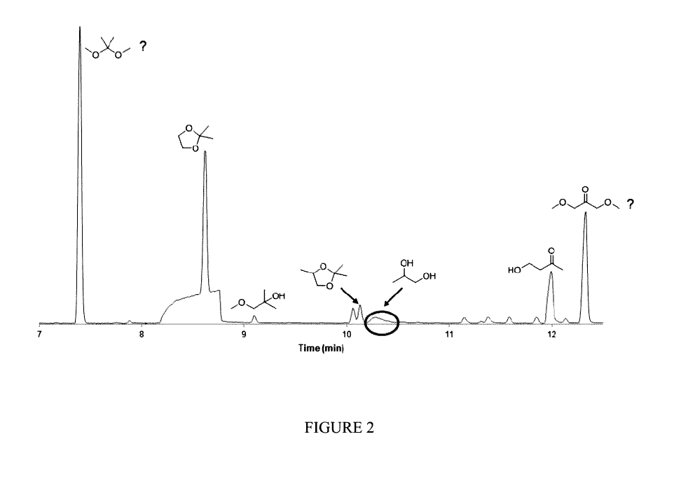

This example illustrates the general procedure for forming ethylene glycol from methanol feed in an autoclave reactor. In a 300 cc autoclave the following were loaded: 71 g of methanol and 48 g of DTBP (trade name Luperox DI from Aldrich Chemicals, 98%). The autoclave was sealed, connected to a gas manifold, and pressurized with 600 psig nitrogen. The reactor content was heated under stirring (500 rpm) at a rate of 2° C./min to 150° C. and held for 4 hours. The heat was turned off and the autoclave allowed to cool down to room temperature. A sample was taken and analyzed by GC analysis, showing complete conversion of DTBP. The autoclave was opened and the reactor content collected at the end of the run, recovering 90% of the materials loaded. The products were analyzed by GC. The run was repeated using a 2-hour hold time. GC-MS traces for the reaction products are shown in FIG. 1 (full range) and in FIG. 2 (zoomed in the region containing ethylene glycol). The GC results are shown below in Table 1, demonstrating that ethylene glycol is produced according to the teachings of the present disclosure:

TABLE 1

| | | |
|---|---|---|
| Reaction temperature (° C.) | 150 | 150 |
| Time (h) | 4 | 2 |
| Methanol, g | 71.0 | 71.1 |
| DTBP, g | 48.1 | 48.0 |
| Oxygenates wt. sel. (%) | | |
| ethylene glycol | 56.4 | 52.4 |
| 2,2-dimethyl-1,3-dioxolane | 15.0 | 14.3 |
| propylene glycol | 3.0 | 3.7 |
| Ethylene glycol mono-acetal | 3.7 | 2.3 |
| 1,3-dioxolane-4-methanol, 2,2-dimethyl | 6.6 | 9.3 |
| 1,3-bis(methoxy) propanone | 9.2 | 8.4 |
| Other oxygenates | 6.1 | 9.6 |

### Additional Embodiments

### Embodiment 1

A process for making di-functional or multi-functional molecules, comprising oxidizing a first feed stream comprising one or more iso-paraffins to form alkyl hydroperoxides and first tertiary alcohols; catalytically converting the alkyl hydroperoxides and first alcohols to dialkyl peroxides; and coupling a second feed stream using the dialkyl peroxides as a radical initiator to create di-functional or multi-functional molecules, while the dialkyl peroxides are converted to second tertiary alcohols.

### Embodiment 2

The process according to embodiment 1, wherein the first feed stream comprises iso-butane.

### Embodiment 3

The process according to embodiment 1, wherein the second feed stream comprises one or more functional molecules of the formula R—(CH₂)ₙ—CHXY; wherein —X and —Y are independently selected functional groups; wherein R is selected from hydrogen, hydrocarbyl, or an independently selected functional group; and wherein n is an integer in the range of 0-30.

### Embodiment 4

The process according to any of the previous embodiments, wherein the one or more functional groups are independently selected from halogens, —OH, —CN, —C(O)OH, —NH, —SH, —NO₂, OSO₃H, —OPO₃H, or —OBOH.

### Embodiment 5

The process according to embodiment 5, wherein the halogens are selected from —F, —Cl, —Br, or —I.

### Embodiment 6

A process for making ethylene glycol, comprising oxidizing a iso-butane to form t-butyl hydroperoxide and t-butyl alcohol; catalytically converting the t-butyl hydroperoxide and the t-butyl alcohol to di-t-butyl peroxide; and coupling methanol into ethylene glycol using the di-t-butyl peroxide as a radical initiator, while the di-t-butyl peroxide is converted to t-butyl alcohol.

### Embodiment 7

A process for making succinic acid, comprising oxidizing a iso-butane to form t-butyl hydroperoxide and t-butyl alcohol; catalytically converting the t-butyl hydroperoxide and the t-butyl alcohol to di-t-butyl peroxide; and coupling acetic acid into succinic acid using the di-t-butyl peroxide as a radical initiator, while the di-t-butyl peroxide is converted to t-butyl alcohol.

### Embodiment 8

A process for making succinonitrile, comprising oxidizing a iso-butane to form t-butyl hydroperoxide and t-butyl alcohol; catalytically converting the t-butyl hydroperoxide and the t-butyl alcohol to di-t-butyl peroxide; and coupling acetonitrile into succinonitrile using the di-t-butyl peroxide as a radical initiator, while the di-t-butyl peroxide is converted to t-butyl alcohol.

### Embodiment 9

A process for making ethylene diamine, comprising oxidizing a iso-butane to form t-butyl hydroperoxide and t-butyl alcohol; catalytically converting the t-butyl hydroperoxide and the t-butyl alcohol to di-t-butyl peroxide; and coupling methyl amine into ethylene diamine using the di-t-butyl peroxide as a radical initiator, while the di-t-butyl peroxide is converted to t-butyl alcohol.

### Embodiment 10

A process for making 1,2-dinitroethane, comprising oxidizing a iso-butane to form t-butyl hydroperoxide and t-butyl alcohol; catalytically converting the t-butyl hydroperoxide and the t-butyl alcohol to di-t-butyl peroxide; and coupling nitromethane into 1,2-dinitroethane using the di-t-butyl peroxide as a radical initiator, while the di-t-butyl peroxide is converted to t-butyl alcohol.

### Embodiment 11

A process for making 1,2-dichloroethane, comprising oxidizing a iso-butane to form t-butyl hydroperoxide and t-butyl alcohol; catalytically converting the t-butyl hydroperoxide and the t-butyl alcohol to di-t-butyl peroxide; and coupling methyl chloride into 1,2-dichloroethane using the di-t-butyl peroxide as a radical initiator, while the di-t-butyl peroxide is converted to t-butyl alcohol.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings therein. it is therefore evident that the particular embodiments disclosed above may be altered or modified and all such variations are considered within the scope and sprit of the present disclosure. Unless otherwise indicated, all numbers expressing quantities of ingredients, properties, reaction conditions, and so forth, used in the specification and claims are to be understood as approximations based on the desired properties sought to be obtained by the present disclosure. Whenever a numerical range with a lower limit and an upper limit is disclosed, a number falling within the range is specifically disclosed. Moreover, the indefinite articles “a” or “an”, as used in the claims, are defined herein to mean one or more than one of the element that it introduces.

1. A process for making di-functional or multi-functional molecules, comprising:
(a) oxidizing a first feed stream comprising one or more iso-paraffins to form alkyl hydroperoxides and first tertiary alcohols;
(b) catalytically converting the alkyl hydroperoxides and first alcohols to dialkyl peroxides; and
(c) coupling a second feed stream using the dialkyl peroxides as a radical initiator to create di-functional or multi-functional molecules, while the dialkyl peroxides are converted to second tertiary alcohols.

2. The process of claim 1, wherein the first feed stream comprises iso-butane.

3. The process of claim 1, wherein the second feed stream comprises one or more functional molecules of the formula R—(CH₂)ₙ—CHXY; wherein —X and —Y are independently selected functional groups; wherein R is selected from hydrogen, hydrocarbyl, or an independently selected functional group; and wherein n is an integer in the range of 0-30.

4. The process of claim 3, wherein the one or more functional groups are independently selected from halogens, —OH, —CN, —C(O)OH, —NH—, —SH, —NO₂, —OSO₃₁H, —OPO₃H, or —OBOH.

5. The process of claim 4, wherein the halogens are selected from —F, —Cl, —Br, or —I.

6. A process for making ethylene glycol, comprising:
(a) oxidizing a iso-butane to form t-butyl hydroperoxide and t-butyl alcohol;
(b) catalytically converting the t-butyl hydroperoxide and the t-butyl alcohol to di-t-butyl peroxide; and
(c) coupling methanol into ethylene glycol using the di-t-butyl peroxide as a radical initiator, while the di-t-butyl peroxide is converted to t-butyl alcohol.

7. A process for making succinic acid, comprising:
(a) oxidizing a iso-butane to form t-butyl hydroperoxide and t-butyl alcohol;
(b) catalytically converting the t-butyl hydroperoxide and the t-butyl alcohol to di-t-butyl peroxide; and
(c) coupling acetic acid into succinic acid using the di-t-butyl peroxide as a radical initiator, while the di-t-butyl peroxide is converted to t-butyl alcohol.

8. A process for making succinonitrile, comprising:
(a) oxidizing a iso-butane to form t-butyl hydroperoxide and t-butyl alcohol;
(b) catalytically converting the t-butyl hydroperoxide and the t-butyl alcohol to di -t-butyl peroxide; and
(c) coupling acetonitrile into succinonitrile using the di-t-butyl peroxide as a radical initiator, while the di-t-butyl. peroxide is converted to t-butyl alcohol.

9. A process for making ethylene diamine, comprising:
(a) oxidizing a iso-butane to form t-butyl hydroperoxide and t-butyl alcohol;
(b) catalytically converting the t-butyl hydroperoxide and the t-butyl alcohol to di-t-butyl peroxide; and
(c) coupling methyl amine into ethylene diamine using the di-t-butyl peroxide as a radical initiator, while the di-t-butyl peroxide is converted to t-butyl alcohol.

10. A process for making 1,2-dinitroethane, comprising:
(a) oxidizing a iso-butane to form t-butyl hydroperoxide and t-butyl alcohol;
(b) catalytically converting the t-butyl hydroperoxide and the t-butyl alcohol to di-t-butyl peroxide; and
(c) coupling nitromethane into 1,2-dinitroethane using the di-t-butyl peroxide as a radical initiator, while the di-t-butyl peroxide is converted to t-butyl alcohol.

11. A process for making 1,2-dichloroethane, comprising:
(a) oxidizing a iso-butane to form t-butyl hydroperoxide and t-butyl alcohol;
(b) catalytically converting the t-butyl hydroperoxide and the t-butyl alcohol to di-t-butyl peroxide; and
(c) coupling methyl chloride into 1,2-dichloroethane using the di-t-butyl peroxide as a radical initiator, while the di-t-butyl peroxide is converted to t-butyl alcohol.
